(19) United States
(12) Patent Application Publication
(10) Pub. No.: US 2026/0026789 A1
(43) Pub. Date: Jan. 29, 2026

(54) CRYOBIOPSY DEVICES, SYSTEMS, AND RELATED METHODS

(71) Applicant: Boston Scientific Scimed, Inc., Maple Grove, MN (US)

(72) Inventor: Ryan V. WALES, Shrewsbury, MA (US)

(73) Assignee: Boston Scientific Scimed, Inc., Maple Grove, MN (US)

(21) Appl. No.: 19/277,513

(22) Filed: Jul. 23, 2025

Related U.S. Application Data
(60) Provisional application No. 63/675,364, filed on Jul. 25, 2024.

(51) Int. Cl. A61B 10/02

(52) U.S. Cl. CPC A61B 10/0233; A61B 2010/0208

(57) ABSTRACT

Medical devices, systems, and methods described herein may be useful for performing a biopsy medical at a target tissue site within a patient and removing a biopsy sample from the target tissue site. The medical device includes a probe that includes a lumen, and a handle in fluid communication with the probe. The handle including a body defining a chamber coupled to the probe and configured to receive a canister containing a fluid, and an actuator configured to transition a valve from a closed position in which the probe is not in fluid communication with the handle and into an open position in which the probe is in fluid communication with the handle.

### CROSS-REFERENCE TO RELATED APPLICATIONS

This application claims the benefit of priority to U.S. Provisional Application No. 63/675,364, filed on Jul. 25, 2024, which is incorporated by reference herein in its entirety.

### TECHNICAL FIELD

Various aspects of the disclosure relate generally to biopsy devices, systems, and related methods. Examples of the disclosure relate to cryobiopsy devices, for example, to perform cryobiopsy medical procedures for extracting biopsy samples, among other aspects.

### BACKGROUND

Advancements in medical devices, systems, and related methods, have enabled users to perform increasingly complex medical procedures, such as biopsy procedures that include the removal and analysis of a body tissue sample (“biopsy sample”) for medical diagnosis. For example, cryobiopsy devices use a cooled tip to acquire a tissue sample. Carbon dioxide may be used to cool the distal tip of such cryobiopsy devices. It would be useful to improve medical devices, systems, and related methods for cryobiopsy.

### SUMMARY

Examples of this disclosure relate to, among other things, medical systems, devices, and methods for cryobiopsy. For example, examples of this disclosure relate to cryobiopsy devices and systems that utilize handheld cartridges of carbon dioxide.

According to one aspect, the disclosure provides a medical device including a probe including a lumen, and a handle. The handle including a body defining a chamber in fluid communication with the lumen of the probe and configured to receive a canister containing a fluid, and an actuator movable relative to the body and configured to transition a valve from a closed position into an open position. In the closed position, the lumen is not in fluid communication with the chamber. In the open position, the lumen is in fluid communication with the chamber.

According to some aspects, the probe may include a distalmost tip of the medical device. The lumen may be a first lumen, wherein the medical device further includes a shaft extending between the handle and the probe, the shaft including a second lumen in fluid communication with the first lumen. An outer wall of the shaft may include at least one exhaust hole in fluid communication with the second lumen, and the at least one exhaust hole may be positioned on the shaft such that it remains outside of a subject's body when the probe contacts a tissue of the subject's body. The at least one exhaust hole may include a plurality of exhaust holes extending radially through the outer wall and into the second lumen. The plurality of exhaust holes may be positioned circumferentially around the outer wall. The chamber may include a complementary engagement feature configured to secure the canister therein, wherein the complementary engagement feature may include an internal thread formed on an inner surface of the chamber that is configured to engage an external thread on an outer surface of the canister. The fluid contained in the canister may be pressurized carbon dioxide.

According to some aspects, the medical device may include a piercing tip disposed within the chamber and is configured to puncture a portion of the canister received therein. The actuator may include a first actuator, and wherein the medical device further includes a second actuator configured to advance the canister toward the piercing tip in order to puncture the canister. The second actuator may be rotatable to advance the canister toward the piercing tip. The actuator may include a living hinge formed in the body and configured to release fluid from the chamber. The actuator may include a button movably disposed in a proximal portion of the body. The actuator may include a stem movably disposed in a lumen defined by the body, where the stem may extend distally through the lumen from the proximal portion of the body to a cavity formed in the body. The actuator may include a first seal positioned at a distal end of the stem and movably disposed within the cavity of the body, and a second seal positioned on the stem longitudinally between the first seal and the proximal portion of the body. The first seal may be configured to seal the lumen in the closed position of the valve, and to allow fluid communication between the lumen and cavity in the open position of the valve in response to actuation of the actuator.

According to another aspect, the disclosure may provide a medical device, including a shaft, and a probe at a distal end of the shaft and defining a probe cavity. The shaft having an outer wall defining an outer lumen, wherein a plurality of exhaust holes extends radially through the outer wall into the outer lumen, the plurality of exhaust holes arranged circumferentially around the outer wall. The medical device including a conduit extending through the outer lumen and into the probe, the conduit defining an inner lumen, wherein a fluid is configured to flow distally through the inner lumen, into the probe cavity, proximally through the outer lumen, and out of the plurality of exhaust holes.

According to some aspects, the medical device may include a handle coupled to the shaft and defining a handle cavity and a handle lumen, wherein the handle includes an actuator movably disposed in the handle lumen, wherein the actuator is configured to selectively place the handle cavity in fluid communication with the inner lumen. The medical device may include a configuration in which the shaft and the probe are disposed within a body lumen of a subject, wherein the plurality of exhaust holes is disposed outside of the body lumen.

According to yet another aspect, the disclosure may provide a medical device handle including a body defining a chamber that is configured to receive an entirety of a fluid canister, a first actuator movable relative to the body, and a second actuator movable relative to the body. The first actuator configured to transition a valve from a closed position into an open position, causing the chamber to release a fluid from the fluid canister into a lumen of a cryogenic probe. The second actuator configured to move the fluid canister distally in order to puncture the fluid canister. According to some aspects, the first actuator of the medical device handle may include a beam and a living hinge, and the second actuator of the medical device handle may be configured to rotate relative to the body to move the fluid canister distally.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and, together with the description, serve to explain the principles of the disclosure.

FIGS. 1A, 1B, and 1C depict various views of a medical device, according to aspects of the disclosure.

FIGS. 2A and 2B depict various views of a medical system, according to aspects of the disclosure.

### DETAILED DESCRIPTION

Examples of the disclosure include devices, systems, and methods for a biopsy instrument that selectively releases refrigerant for freezing tissue at a target tissue site within a patient and removing a biopsy sample from the target tissue site. The disclosed devices may be coupled to a canister or cartridge of carbon dioxide (e.g., a canister or cartridge that is capable of being held in a single hand of a user). The disclosed devices may include mechanisms to receive and/or couple the cartridge or canister to the devices. For example, a handle of a medical device may include a chamber for receiving a canister or cartridge. Alternatively, a handle of a medical device may include a coupler for screwing onto or otherwise coupling to a canister or cartridge, such that a user may lift and/or otherwise hold an entirety of the canister or cartridge when it is coupled to the handle. The handle may lack tubing or the like that couples to a source of fluid for cooling the probe.

As used herein, the term “distal” refers to a portion farthest away from a user when introducing a device into a patient and the term “proximal” refers to a portion closest to the user when placing the device into the subject. The terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to system, devices, and methods for performing various medical procedures and/or treating portions of the biliary duct, large intestine, small intestine, cecum, esophagus, any other portion of the gastrointestinal tract, and/or any other suitable patient anatomy (collectively referred to herein as a “target site”). The devices disclosed herein may be entirely disposable, entirely reusable, or may include a combination of disposable and reusable devices. Reference will now be made in detail to examples of the disclosure described above and illustrated in the accompanying drawings. Wherever possible, the same reference numbers will be used throughout the drawings to refer to the same or like parts.

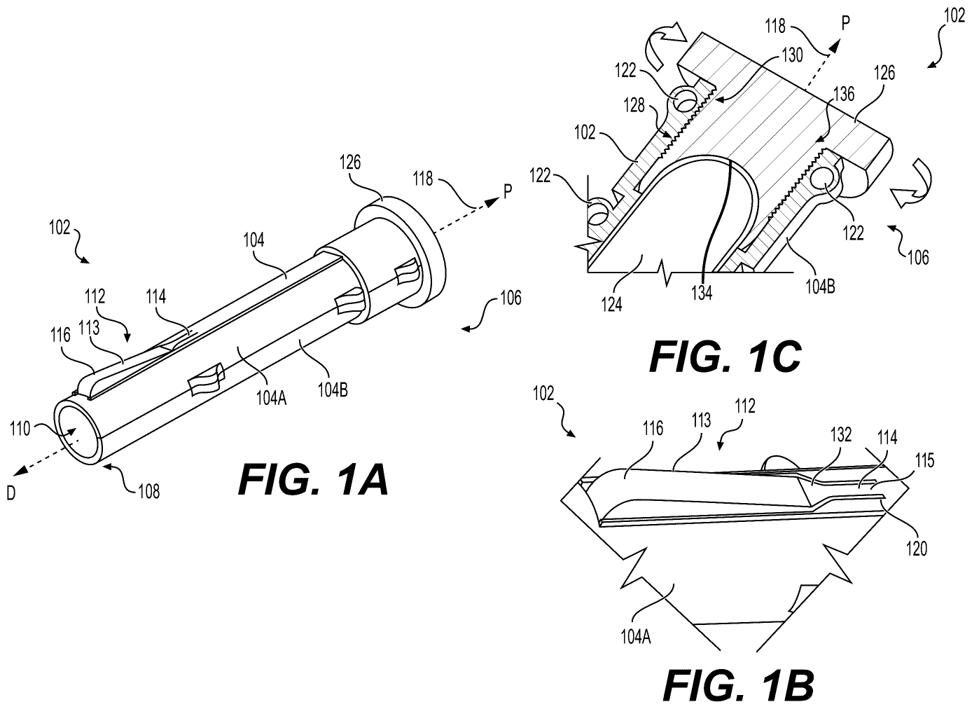

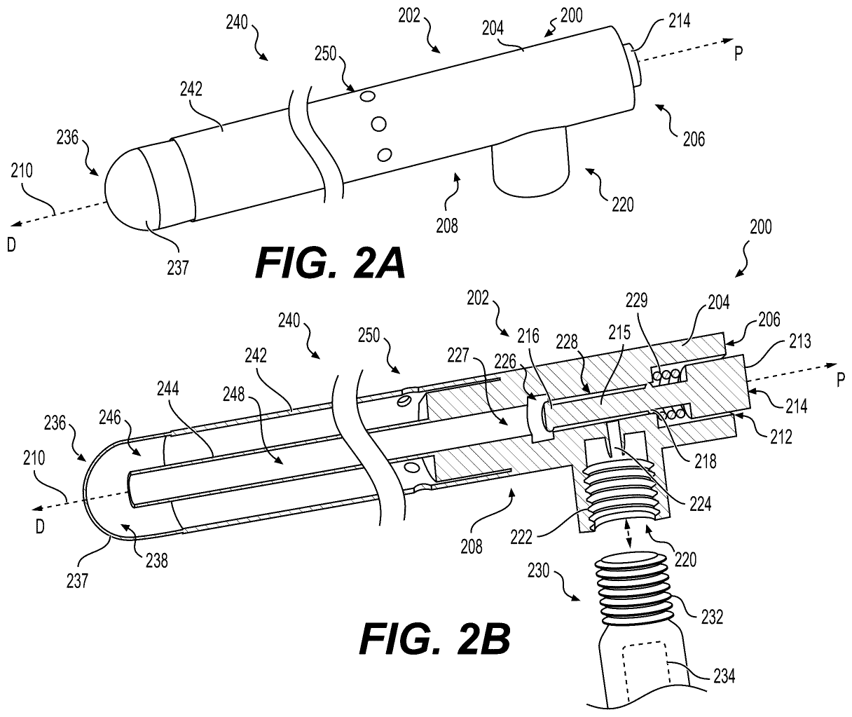

FIGS. 1A-1C illustrate a handle 102 of a medical device, according to aspects of the disclosure. As discussed herein, handle 102 is configured to transmit a fluid through one or more conduits of a shaft in fluid communication with handle 102, and through one or more probe conduits of one or more cryogenic probes (hereinafter, “probe”) in fluid communication with the shaft. Although such a probe is not shown in FIGS. 1A-1C, the probe may have any of the features depicted in FIGS. 2A and 2B and described below. The fluid acts as refrigerant flowing through the probe conduit, causing a rapid change in temperature of the probe and via the Joule-Thompson effect. When a distal tip of the probe is placed in contact with tissue at a target site, the probe freezes tissue in contact with the probe. The frozen tissue may then be extracted to yield a cryobiopsy biopsy sample from the target site. Although not shown, the probe and/or the shaft coupled to handle 102 may be delivered through a working channel or other lumen of an endoscope, duodenoscope, gastroscope, colonoscope, ureteroscope, bronchoscope, and/or other delivery systems to the target site.

A fluid canister may be coupled to handle 102 in order to cool the distal tip of the probe. Although the fluid canister is not depicted in FIGS. 1A-1C, the fluid canister depicted in FIGS. 2A-2B and described below may be utilized with the device of FIGS. 1A-1C. For example, the canister may house pressurized carbon dioxide (CO₂). However, a variety of fluids may be used as refrigerant to cool the probe fluidly coupled to handle 102, such as gaseous refrigerant, liquid refrigerant, or super-critical refrigerants. In some examples, the fluid includes another pressurized gas, such as nitrous oxide (NO₂). When released from the canister, the pressurized gas may achieve cooling of the probe by expansion of the gas and the Joule-Thomson effect.

As discussed herein, handle 102 is shaped and dimensioned to receive at least a portion of at least one canister containing at least one fluid (e.g., for cooling the probe during medical procedures). In some examples, handle 102 may be configured to accommodate one or more different sized canisters containing fluid therein, such as an 8-gram canister, 12-gram canister, 16-gram canister, 20-gram canister, etc. The canister may be a size such than a user may hold the handle including the canister. For example, the user may lift handle 102 and an entirety of the canister. The canister may contrast with a large canister that must be stored on a floor or in another storage location or with other types of capital equipment. Handle 102 may not include any tubing or the like that couples handle 102 to an external source of fluid for cooling a probe.

FIG. 1A illustrates a perspective view of handle 102. As shown, handle 102 includes a body 104 extending along a longitudinal axis 118 between a proximal portion 106 and a distal portion 108. Body 104 includes one or more inner surfaces defining a lumen 110 of handle 102. In examples, lumen 110 may receive or otherwise be coupled to a probe or a shaft that terminates in a probe. Lumen 110 may be in fluid communication with a lumen of a probe of the medical device, such that gas from the canister may be delivered from lumen 110 into the lumen of the probe.

In some examples, handle 102 includes two or more portions coupled together to define body 104. For example, body 104 of handle 102 may be split longitudinally into a first portion 104A and a second portion 104B, which are coupled together to form body 104 at complementary surfaces. As shown in FIG. 1C, body 104 includes one or more apertures 122 configured to receive one or more fasteners therein (not shown), e.g., for coupling first portion 104A and second portion 104B together to assemble body 104 of handle 102. In one example, apertures 122 extend through complementary surfaces in each of first portion 104A and second portion 104B of body 104, such that the fasteners (e.g., one or more pins) are shaped and dimensioned to extend through apertures 122 in each of first portion 104A and second portion 104B to assemble body 104 of handle 102. It should be understood that other fasteners may be alternatively or additionally used. In other examples handle 102 may be unitary or integrally formed, for example by using one or more additive manufacturing and/or reductive manufacturing processes to define the shape and dimensions of body 104 of handle 102. In further examples, body 104 may include a separate proximal and distal portion that may be coupled together.

In some aspects, body 104 may be segmented to define two or more portions formed therein that are in fluid communication with each other. For example, as shown in FIG. 1C, body 104 defines a chamber 124 positioned at proximal portion 106 of handle 102. Chamber 124 may be shaped and dimensioned to receive a canister therein. In one example, handle 102 receives the canister through a proximal opening 136 formed in proximal portion 106 of body 104 and extending into chamber 124. In another example, complementary first portion 104A and second portion 104B defining body 104 at least partially separate to allow the canister to be slidably received in chamber 124 therein. For instance, handle 102 may include a hinge (not shown) pivotably coupling complementary first portion 104A and second portion 104B defining body 104, such that at least one complementary portion of body 104 moves relative to another portion and thus allows passage of the canister into chamber 124. In yet another example, handle 102 receives the canister through a distal opening formed in distal portion 108 of body 104 (e.g., a distal opening of lumen 110). In aspects, an entirety of the canister may be received within the chamber 124 or within other portions of handle 102. The entirety of the canister may be substantially enclosed within the chamber 124 or other portions of handle 102.

Handle 102 (e.g., body 104) may include one or more features configured to puncture one or more portions of the canister received therein. In some examples, handle 102 includes a piercing tip (not shown but having any property of piercing tip 224, described below) disposed at least partially in chamber 124 and/or distally to chamber 124. The piercing tip may be configured to puncture a portion of the canister received therein (e.g., to release fluid contained in the canister into the chamber). The piercing tip may be positioned proximate to the canister within the chamber. For example, the piercing tip may be disposed at a distalmost end of chamber 124 and may be configured to pierce a distal end of the canister (e.g., an end of a neck of the canister). The piercing tip may be similar to a needle, lance, or other protruding structure.

Handle 102 may include at least one actuator 126 that is movable relative to body 104 and the piercing tip, such that the actuator moves the canister into contact with the piercing tip to release fluid contained in the canister (e.g., into chamber 124 and/or lumen 110).

Actuator 126 that is movable relative to body 104 (e.g., distally, or proximally along axis 118). Actuator 126 includes a thread 128 (e.g., an external thread) for complementary engagement to a thread 130 (e.g., an internal thread 130) formed on an inner surface of body 104 at the proximal end of body 104. Alternatively, actuator 126 may include an internal thread, and body 104 may include an external thread. Actuator 126 is rotatable (e.g., clockwise, and/or counter-clockwise) relative to body 104. For example, actuator 126 may align with proximal opening 136 formed in proximal portion 106 of body 104 along axis 118, such that rotating actuator 126 in a first direction (e.g., clockwise) moves actuator 126 distally relative to body 104 along axis 118. Rotating actuator 126 in a second direction (e.g., counter-clockwise) moves actuator 126 proximally through chamber 124 along axis 118. In some examples, actuator 126 may be removed entirely from body 104 so that a canister may be inserted into proximal opening 136. Actuator 126 may then be inserted into proximal opening 136 and rotated in order to advance actuator 126 distally.

Actuator 126 includes at least one surface shaped and dimensioned to engage the canister disposed in chamber 124, such that actuator 126 is configured to move the canister distally within chamber 124. For example, a distal surface 134 of actuator 126 may contact a proximal end of the canister, such that distal movement of actuator 126 causes distal movement of the canister, which may bring a distalmost end of the canister (e.g., an end of the canister that is opposite the distal surface 134) into contact with the piercing tip, thereby puncturing the canister and releasing fluid contained therein. In one example, rotating actuator 126 in the first direction (e.g., clockwise) relative to body 104 causes actuator 126 to move relative to body 104 (e.g., distally along axis 118), and in turn causes the canister to move into contact with the piercing tip, puncturing the canister and releasing fluid contained therein. Distal surface 134 may have a shape that complements (is a negative of) a shape of a proximal end of the canister. For example, distal surface 134 may have a concave shaped configured to be complementary to a domed proximal shape of the canister.

In alternative examples, the piercing tip may be movable relative to the chamber such that the piercing tip may move into contact with and puncture the portion of the canister. For instance, the piercing tip may be movably coupled to an actuator at least partially disposed in body 104 (e.g., in chamber 124), such that actuating the actuator causes the piercing tip to move (e.g., translate) through and relative to chamber 124 into contact with the canister received therein. In some examples, the piercing tip may extend radially through, and movable relative to, body 104 at a non-zero angle relative to axis 118. In other examples, the piercing tip is fixed to an inner surface defining chamber 124 of handle 102.

In another example, actuator 126 includes at least one sharp portion movable relative to the canister disposed therein, such that actuator 126 is configured to move into contact and puncture a portion of the canister. For instance, distal surface 134 of actuator 126 may include at least one piercing tip configured to puncture the canister (e.g., as actuator 126 is advanced distally relative to body 104).

In alternative implementations, actuator 126 may be slidably disposed in one or more slots formed in proximal portion 106 of body 104. In this example, actuator 126 may slide proximally or distally through the slot(s) formed in body 104, causing the canister to move distally through chamber 124 into contact with the piercing tip disposed therein. Handle 102 may include a locking mechanism, such as a locking pin slidably disposed at least partially in the slot(s), configured to engage actuator 126 within the slot(s) and inhibit proximal and/or distal movement relative to body 104.

Chamber 124 may be coupled to lumen 110 via one or more components, such as valves and/or seals (not shown but having any of the features of any known valves or seals) disposed in body 104 and selectively fluidly coupling chamber 124 with lumen 110. Handle 102 may include one or more actuators 112 operably coupled to the valves and/or seals, such that the actuators 112 are configured to selectively release or transmit fluid contained within chamber 124 into lumen 110. Handle 102 therefore may be configured to contain fluid (e.g., pressurized gas) within chamber 124, and configured to selectively release fluid from chamber 124 and through lumen 110 toward distal portion 108 of handle 102, upon actuation of actuator 112.

For example, actuator 112 may include a beam 113 coupled to a remainder of body 104 by a living hinge 115 (labeled in FIG. 1B). Beam 113 may extend between a proximal portion 114 of beam 113 and a distal portion 116 of beam 113. Beam 113 may extend through a slot 120 extending longitudinally (e.g., parallel to axis 118) through a wall of body 104. Beam 113 may be integrally formed with a material of body 104 or a different material and may be coupled to a remainder of body at living hinge 115.

Actuator 112 (e.g., beam 113) may have a variable radial width (along an axis perpendicular to axis 118 and intersecting axis 118) or cross-section along a length thereof. For example, proximal portion 114 may have a first width, and distal portion 116 may have a second width greater than the first width. Beam 113 may include a middle portion 132 that is tapered and transitions from the first width at proximal portion 114 to the second width at distal portion 116. The greater radial width of distal portion 116 may facilitate contact and actuation (e.g., pressing distal portion 116 radially inward), and the smaller radial width of proximal portion 114 may facilitate bending of beam 113 at living hinge 115.

As mentioned above, in some aspects, distal portion 116 of actuator 112 is movable in at least one direction (e.g., radially-inward) relative to proximal portion 114 and/or middle portion 132 of actuator 112. In an unactuated position, distal portion 116 of actuator 112 may flare or extend radially-outward relative to proximal portion 114 and/or middle portion 132. In some aspects, actuator 112 may include a flexible material which allows movement of proximal portion 114, distal portion 116, and/or middle portion 132 relative to other portions of actuator 112 while remaining affixed to body 104 via proximal portion 114). Alternatively, beam 113 may be formed of a same rigid material as a remainder of body 104, and a radial thinness of living hinge 115 may allow for beam 113 to flex at living hinge 115. Actuating actuator 112 (e.g., radially depressing distal portion 116) may cause handle 102 to release fluid contained within chamber 124, such as to lumen 110 and to the shaft and probe in fluid communication with handle 102.

In some examples, actuating actuator 112 causes distal portion 116 to move into contact with one or more valves of handle 102, which in turn causes the valve(s) to move into an open position and release fluid from chamber 124 into lumen 110. In these examples, releasing actuator 112 may cause distal portion 116 to disengage the valve(s), returning the valve(s) to a closed position to seal the chamber 124 from lumen 110. For example, actuator 112 may have shape memory properties.

In alternative implementations, actuating actuator 112 may drive the piercing tip(s) into contact with the canister, causing the canister to release the entire volume of fluid contained therein.

In alternative implementations, actuator 112 may include a button, slide, or other component configured to release the refrigerant from at least one canister within handle 102. Handle 102 may include a biasing member (e.g., a spring) positioned within body 104 configured to bias the button, slide, or other component configured to release refrigerant from at least one canister (or chamber) within handle 102.

In some examples, the user inserts the canister containing pressured gas (e.g., carbon dioxide) into chamber 124 of handle 102 via proximal opening 136. The user may then insert actuator 126 into proximal opening 136 and rotate actuator 126 to distally advance actuator 126 relative to body 104, such that distal surface 134 moves into contact with a proximal end of the canister therein. Further rotating actuator 126 causes the canister to move within chamber 124 and into contact with the piercing tip configured to puncture at least one portion of the canister to release pressured gas contained therein. The user actuates actuator 112 to transmit gas from chamber 124 and distally through lumen 110. The fluid flows through lumen 110 and to a probe (e.g., coupled to a distal end of a shaft), causing the probe to rapidly cool to perform a cryobiopsy procedure at the target site in contact with an outer surface of the probe. The probe may be retracted relative to the target site, causing frozen tissue to adhere to the outer surface of the probe for removal from the subject's body.

FIGS. 2A-2B illustrates a medical device 200 including a cryogenic probe 236 (hereinafter, “probe 236”), a handle 202, and a shaft 240 extending between handle 202 and probe 236. Medical device 200 may be useful, for example, to capture a biopsy sample from a tissue of a subject (e.g., a human, pet, livestock, etc.). For example, probe 236 may be inserted into the subject and placed into contact with a target tissue. A fluid (e.g., a gas from a canister 230) may be introduced into probe 236, forming a region of cooled tissue in contact with an outer surface of probe 236, in turn causing cooled tissue to adhere to probe 236. After cooling tissue, probe 236 is withdrawn along with a biopsy sample coupled with probe 236.

As shown, handle 202 includes a body 204 that extends between a proximal portion 206 and a distal portion 208 along a longitudinal axis 210. Handle 202 may define a grip portion configured to be gripped by the user's hand during medical procedures, such as at proximal portion 206 of body 204. Distal portion 208 of handle 202 may be coupled to a proximal end of shaft 240, such that at least one conduit or lumen defined by handle 202 is in fluid communication with at least one conduit of shaft 240, as further discussed herein.

FIG. 2B illustrates a cross-sectional view of medical device 200 according to aspects of the disclosure. As shown, body 204 defines a port or chamber 220 of handle 202 configured to receive a canister 230 containing a volume of fluid 234 therein. Chamber 220 includes an internal thread 222 configured to threadably engage an external thread 232 of canister 230. To assemble medical device 200, the user may align canister 230 with an opening of chamber 220 of handle 202, and rotate canister 230 to secure canister 230 in chamber 220 by threads 222 and 232. Chamber 220 is in fluid communication with at least one conduit of shaft 240, as further discussed herein.

In some examples, handle 202 may include a piercing tip 224 positioned in chamber 220. Piercing tip 224 may be positioned proximate internal thread 222, such that rotating canister 230 causes threaded advancement of canister 230 through chamber 220 and into contact with piercing tip 224. Piercing tip 224 may be shaped and dimensioned to puncture a portion of canister 230, causing pressurized gas to flow out of canister 230.

Body 204 includes one or more inner surfaces defining a proximal, first lumen 228 of handle 202. First lumen 228 may extend substantially parallel to axis P and approximately perpendicularly to a longitudinal length of chamber 220. A distal, second lumen 227 defined by body 204 may be selectively fluidly coupled to first lumen 228 by a cavity 226, which may be between first lumen 228 and second lumen 227. Second lumen 227 may extend approximately parallel to axis P. Further details of first lumen 228, cavity 226, and second lumen 227 are provided below. In examples, second lumen 227 may be coupled to an inner lumen 248 of a conduit 244 of shaft 240, described below, such that gas from canister 230 may be selectively delivered from chamber 220, into lumens 228 and 227, and to probe 236 as described below

Probe 236 includes an outer wall 237, an inner surface of which may define a lumen or cavity 238. Shaft 240 includes an outer wall 242 defining an outer lumen 246. Conduit 244 (e.g., a tube) may extend through outer lumen 246 of shaft 240 and into cavity 238 of probe 236. Conduit 244 may terminate proximally of a distal end of probe 236. Conduit 244 may define inner lumen 248. A distal end of inner lumen 248 may be open, such that inner lumen 248 is in fluid communication with cavity 238 of probe 236. Cavity 238 may be distal of and in fluid communication with outer lumen 246 of shaft 240. Inner lumen 248 may be in fluid communication with second lumen 227 of handle 202.

Shaft 240 may include one or more exhaust holes 250 extending through outer wall 242. As shown, exhaust holes 250 may be positioned circumferentially around shaft 240, extending radially through outer wall 242 and into outer lumen 246. It should be understood that exhaust holes 250 may include more or fewer holes formed in shaft 240 and/or may be arranged in different positions along outer wall 242. Exhaust holes 250 may be formed in a portion of shaft 240 that remains external to a subject's body during a medical procedure. During medical procedures, fluid may flow distally from chamber 220, into lumen 228, into lumen 227, into inner lumen 248, out of a distal opening of conduit 244 into cavity 238, proximally through outer lumen 246, and through exhaust holes 250 into an environment/atmosphere. In some examples, inner lumen 248 may be a supply lumen, and outer lumen 246 may be a return lumen. Outer lumen 246 may vent through exhaust holes 250, which may be external to (outside of) a subject's body.

In some examples, medical device 200 includes one or more insulation layers, such as an insulation jacket disposed around one or more portions of handle 202 and/or shaft 240. The insulation jacket may inhibit handle 202 from becoming cold during cryobiopsy medical procedures, such that the user can grasp handle 202 without regard for temperature changes (e.g., due to refrigerant flowing therein). The insulation jacket may inhibit portions of shaft 240 that are proximal to probe 236 from being so cold that they damage tissues of a body lumen.

In some aspects, when handle 202 and canister 230 are assembled for cryobiopsy procedures, actuating one or more actuators 214, described below, of handle 202 causes chamber 220 to selectively be placed in fluid communication with second lumen 227. Actuating the actuator(s) 214 of handle 202 may cause the actuator(s) 214 to move between a closed configuration that seals chamber 220 from second lumen 227, and an open configuration in which chamber 220 is in fluid communication with second lumen 227. Actuator 214 may thus function as a valve. Alternatively, actuator 214 may be separate from a valve and may function to open/close a separate valve.

As shown, a proximal portion 213 of actuator 214 may be movably disposed in a recess 212 formed in proximal portion 206 of body 204. Recess 212 may be in fluid communication with lumen 228 and may have a larger diameter/width than lumen 228. Thus, proximal portion 213 may be slidable within recess 212 but may not move into lumen 228 because a diameter/width of lumen 228 is smaller than a diameter/width of proximal portion 213. Proximal portion 213 may be contacted by an operator and may function as a button that may be depressed by the operator. As described below, distally translating actuator 214 through recess 212 relative to proximal portion 206 causes chamber 220 to release fluid into lumen 228.

Actuator 214 may include a stem 215 (e.g., a valve stem) extending distally from proximal portion 213 through lumen 228 of handle 202. In the example shown in FIG. 2B, stem 215 extends between a proximalmost end coupled to a distalmost end of proximal portion 213, and a distal end movably disposed in cavity 226 defined by body 204. Cavity 226 may be wider (may have a greater cross-section) than lumens 228 and 227.

Actuator 214 may include a distal seal 216 positioned at the distal end of stem 215 (e.g., within cavity 226), and a proximal seal 218 positioned along stem 215 proximal of distal seal 216 and within first lumen 228. Distal seal 216 and proximal seal 216 may be integrally formed with step 215 or may be separate components (e.g., O-rings). Distal seal 216 may be configured to selectively seal cavity 226 from lumen 228, thereby preventing fluid communication between chamber 220 and cavity 226 and second lumen 227. Proximal seal 218 may be configured to seal lumen 228 from recess 212, thereby preventing fluid communication between chamber 220 and recess 212 (and proximal flow of pressurized fluid through handle 202).

In a first, proximal, closed configuration of actuator 214, distal seal 218 may abut a proximal surface of cavity 226. Distal seal 218 may have a greater width/diameter than lumen 228 and a smaller width/diameter than cavity 226. Thus, in the first, proximal, closed configuration, fluid may not pass distally from lumen 228 into cavity 226. Actuating actuator 214 (moving actuator 214 distally) causes stem 215 to advance distally through lumen 228, such that distal seal 216 moves distally relative to and within cavity 226. In a second, distal, open configuration of actuator 214, fluid may be able to flow into cavity 226, around distal seal 218, and into second lumen 227. In other words, as stem 215 advances distally through lumen 228, the distal seal 216 is separated from a distal opening of lumen 228, which allows fluid (e.g., pressurized gas) from chamber 220 to flow through cavity 226 (e.g., into second lumen 227 and into lumen 248 of shaft 240).

Proximal seal 218 may be secured along stem 215 such that distally advancing stem 215 causes proximal seal 218 to slide through lumen 228. Proximal seal 218 may have properties (e.g., diameter/width and material) such that proximal seal 218 inhibits flow from first lumen 228 into recess 212 in both the first, proximal, sealed configuration and the second, distal, open configuration.

Thus, these movable features of actuator and passages in fluid communication collectively define a valve of handle 202 which moves from at least the closed position (e.g., sealing pressurized gas in chamber 220) and the open position (e.g., releasing pressurized gas from chamber 220 and distally into lumen 248).

In some examples, handle 202 may include a spring valve positioned between chamber 220 and lumen 228, such that actuator 214 is configured to move the spring valve relative to the chamber 220 and/or lumen 228. For instance, stem 215 of actuator 214 may have a shape and dimension configured to engage one or more spring valves positioned at least partially within the lumen 228, such that stem 215 may contact and displace the spring valve(s) to move at least from a closed position (e.g., to seal pressurized gas within chamber 220) into an open position (e.g., to release pressurized gas from chamber 220). Stem 215 may include a radial protrusion that slides into contact with a movable portion of the spring valve, such that the radial protrusion causes the spring valve to move into the open position as stem 215 slides through lumen 228 in response to actuation (e.g., depression) of actuator 214.

In some examples, handle 202 may include one or more biasing components configured to bias actuator 214 relative to body 204. In these examples, actuating actuator 214 releases fluid from chamber 220 and, when actuator 214 is released, the biasing component(s) proximally displaces actuator 214 along axis 210 relative to body 204, proximally displacing distal seal 216 within cavity 226 and sealing first lumen 228 from second lumen 227. In other words, actuator 214 may be biased to the first, proximal, sealed configuration. In the example shown in FIG. 2B, handle 202 includes a spring 229 positioned in recess 212, such that spring 229 is configured to bias actuator 214 proximally along axis 210 (e.g., into the closed position). Spring 229 may include, for example, a compression spring positioned within recess 212 and configured to abut or contact a distal surface of proximal portion 213 (e.g., when radially depressing actuator 214). In another example, handle 202 includes the biasing component(s) within cavity 226, for example such that spring 229 contacts distal seal 216 to bias actuator 214 relative to body 204 along axis 210 (e.g., into the closed position).

In alternative implementations, handle 202 may include a trigger actuator movable relative to body 204, such that squeezing the trigger actuator (e.g., proximally) causes handle 202 to move from at least the closed (sealed) position into the open (unsealed) position. Releasing the trigger actuator causes handle 202 to move back into the closed position (e.g., to stop flow of fluid therein). The trigger actuator may be biased toward the closed position of handle 202 via biasing components (e.g., springs). Handle 202 may include a trigger pin that moves relative to body 204 between a locked position configured to engage the trigger actuator preventing movement thereof, and an unlocked position configured to disengage the trigger actuator allowing movement thereof. For example, handle 202 may include the trigger pin slidably disposed in an aperture formed in body 204 proximate the trigger actuator.

In alternative implementations, medical device 200 includes a bolt positioned in a slot formed through body 204. The bolt may include a first end coupled to the sharp portion(s) within the lumen and opposite a second end extending radially from body 204 through the slot. The user (e.g., physician) may grasp the second end of the bolt to slide the bolt proximally and/or distally through the slot relative to body 204. For example, the user may move the second end of the bolt proximally through the slot, such that the first end having the sharp portion(s) is driven proximally into contact with the canister to release refrigerant during medical procedures.

Medical devices, systems, and related methods discussed herein may be useful for performing cryobiopsy medical procedures to acquire a biopsy sample. Assembling medical devices described herein may include loading a cartridge containing fluid therein into a handle of the medical device. The cartridge may be threaded into engagement with the handle and driven into contact with a piercing tip which punctures the canister disposed therein. Puncturing the canister causes fluid, such as pressurized CO₂, to be released into the chamber of the handle. Actuating the handle (e.g., via one or more actuators) causes the handle to transmit fluid from the chamber and through a lumen of a shaft coupled to the handle. Fluid flows from the handle through the lumen of the shaft, and through a lumen or cavity of a probe coupled to the shaft opposite the handle. The fluid may flow from the handle, through a first lumen of the shaft, through the cavity of the probe, through a second lumen of the shaft, and exhaust through at least one exhaust hole extending into the second lumen of the shaft. The fluid rapidly cools the probe (e.g., due to rapid expansion of pressurized gas and the Joule-Thompson throttling effect), such that tissue at a target site of patient anatomy rapidly cools and adheres to an exterior surface of the probe. Moving the medical device away from the target site (e.g., pulling the probe) severs tissue that adheres to the exterior surface of the probe, which may then be removed from the subject's body for subsequent analysis.

Each of the aforementioned devices, systems, and methods may be used for medical procedures to acquire a biopsy sample from a patient. By providing a medical device capable of acquiring biopsy samples using a canister, known problems associated with biopsy medical procedures and/or other aspects of invasive surgical procedures may be reduced or avoided. Based on these aspects, physicians, or other users of may reduce the overall procedure time, increase efficiency of procedures, and/or avoid unnecessary harm to patient anatomy during procedures that involve acquiring biopsy samples.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device, comprising:
a probe including a lumen;
a handle, including:
a body defining a chamber in fluid communication with the lumen of the probe and configured to receive a canister containing a fluid,
an actuator movable relative to the body and configured to transition a valve from a closed position into an open position, wherein, in the closed position, the lumen is not in fluid communication with the chamber and wherein, in the open position, the lumen is in fluid communication with the chamber.

2. The medical device of claim 1, wherein the lumen is a first lumen, the medical device further comprising:
a shaft extending between the handle and the probe,
wherein the shaft includes a second lumen in fluid communication with the first lumen,
wherein an outer wall of the shaft includes at least one exhaust hole in fluid communication with the second lumen, and
wherein the at least one exhaust hole is positioned on the shaft such that it remains outside of a subject's body when the probe contacts a tissue of the subject's body.

3. The medical device of claim 2, wherein the at least one exhaust hole comprises a plurality of exhaust holes extending radially through the outer wall and into the second lumen.

4. The medical device of claim 3, wherein the plurality of exhaust holes is positioned circumferentially around the outer wall.

5. The medical device of claim 2, wherein the probe is a distalmost tip of the medical device.

6. The medical device of claim 1, wherein the chamber comprises a complementary engagement feature configured to secure the canister therein, wherein the complementary engagement feature comprises an internal thread formed on an inner surface of the chamber that is configured to engage an external thread on an outer surface of the canister.

7. The medical device of claim 1, wherein the fluid contained in the canister is pressurized carbon dioxide.

8. The medical device of claim 1, further comprising a piercing tip disposed within the chamber and that is configured to puncture a portion of the canister received therein.

9. The medical device of claim 8, wherein the actuator is a first actuator, and wherein the medical device further comprises a second actuator configured to advance the canister toward the piercing tip in order to puncture the canister.

10. The medical device of claim 9, wherein the second actuator is rotatable to advance the canister toward the piercing tip.

11. The medical device of claim 1, wherein the actuator comprises a living hinge formed in the body and configured to release fluid from the chamber.

12. The medical device of claim 1, wherein the actuator comprises a button movably disposed in a proximal portion of the body.

13. The medical device of claim 12, wherein the actuator comprises a stem movably disposed in a lumen defined by the body, wherein the stem extends distally through the lumen from the proximal portion of the body to a cavity formed in the body.

14. The medical device of claim 13, wherein the actuator comprises a first seal positioned at a distal end of the stem and movably disposed within the cavity of the body, and a second seal positioned on the stem longitudinally between the first seal and the proximal portion of the body.

15. The medical device of claim 14, wherein the first seal is configured to seal the lumen in the closed position of the valve, and to allow fluid communication between the lumen and cavity in the open position of the valve in response to actuation of the actuator.

16. A medical device, comprising:
a shaft having an outer wall defining an outer lumen, wherein a plurality of exhaust holes extends radially through the outer wall into the outer lumen, the plurality of exhaust holes arranged circumferentially around the outer wall;
a probe at a distal end of the shaft and defining a probe cavity; and
a conduit extending through the outer lumen and into the probe, the conduit defining an inner lumen;
wherein a fluid is configured to flow distally through the inner lumen, into the probe cavity, proximally through the outer lumen, and out of the plurality of exhaust holes.

17. The medical device of claim 16, further comprising a handle coupled to the shaft and defining a handle cavity and a handle lumen, wherein the handle comprises an actuator movably disposed in the handle lumen, wherein the actuator is configured to selectively place the handle cavity in fluid communication with the inner lumen.

18. The medical device of claim 16, wherein, in a configuration in which the shaft and the probe are disposed within a body lumen of a subject, the plurality of exhaust holes is disposed outside of the body lumen.

19. A medical device handle, comprising:
a body defining a chamber that is configured to receive an entirety of a fluid canister,
a first actuator movable relative to the body and configured to transition a valve from a closed position into an open position, causing the chamber to release a fluid from the fluid canister into a lumen of a cryogenic probe; and
a second actuator movable relative to the body and configured to move the fluid canister distally in order to puncture the fluid canister.

20. The medical device handle of claim 19, wherein the first actuator comprises a beam and a living hinge, and wherein the second actuator is configured to rotate relative to the body to move the fluid canister distally.
